(19) United States
(12) Patent Application Publication
(10) Pub. No.: US 2015/0208925 A1
(43) Pub. Date: Jul. 30, 2015

(54) Photoacoustic Needle Insertion Platform

(71) Applicant: Actuated Medical, Inc., Bellefonte, PA (US)

(72) Inventors: Roger B Bagwell, Bellefonte, PA (US); Kevin A. Snook, State College, PA (US); Ryan S. Clement, State College, PA (US); Andrew J. Meehan, Warriors Mark, PA (US)

(73) Assignee: Actuated Medical, Inc., Bellefonte, PA (US)

(21) Appl. No.: 14/601,784

(22) Filed: Jan. 21, 2015

Related U.S. Application Data
(60) Provisional application No. 61/931,286, filed on Jan. 24, 2014.

(51) Int. Cl. A61B 5/00; A61B 10/02

(52) U.S. Cl. CPC A61B 5/0095; A61B 10/0233; A61B 5/4887

(57) ABSTRACT

A device for differentiating tissue is provided that has a first laser transmission source that outputs a first laser beam in which output from the first laser transmission source is transferred into tissue. A second laser transmission source is provided that outputs a second laser beam that has a wavelength that is different than the first laser beam. Output from the first and second laser transmission sources is transferred into the tissue. A needle system is present for insertion into the tissue along with an acoustic receiver that receives acoustic waves that are created upon the transfer of the output of the first and second laser transmission sources into the tissue. An associated method is also provided.

### CROSS REFERENCE TO RELATED APPLICATIONS

This patent application claims the benefit of U.S. Patent Application Ser. No. 61/931,286, filed Jan. 24, 2014 entitled Photoacoustic Needle Insertion Platform. U.S. Patent Application Ser. No. 61/931,286 is incorporated by reference herein in its entirety for all purposes.

### STATEMENT REGARDING FEDERALLY SPONSORED RESEARCH OR DEVELOPMENT

This invention was produced in part using funds from the Federal government under National Institutes of Health Contract Award ID No. HHSN261201400015C. Accordingly, the government has certain rights in the invention.

### BACKGROUND OF THE INVENTION

1. Field of the Invention

The present invention pertains generally to the field of medical devices, and more specifically to a photoacoustic system for in-situ characterization and differentiation of biological tissues and fluids during medical procedures and examinations. The invention may incorporate transcutaneous needles where differentiation provides real-time benefits to a health care provider such as, but not limited to, improved needle tip localization, trajectory alignment, targeting or selection of specific structures, or providing feedback before triggering the throw of a biopsy collection tool during a biopsy procedure.

2. Background

The following is a description of the background of core needle biopsies (CNBs) and regional anesthesia (RA) procedures, an example of which is peripheral nerve blocks (PNBs). It should be understood that the device and method of the present invention is not limited to CNBs and RA procedures, but is applicable to a range of transcutaneous needle procedures, such as amniocentesis and pericardial access, and that CNBs and PNBs are being discussed simply by way of example. It should also be understood that the device and method of the present invention is applicable, but not limited to cancer or ligamentous tissues, blood, fatty tissues, lymph, bone, and foreign bodies.

Cancer diagnosis has recently undergone a significant advancement through the use of biomarker profiling. This technique uses ribonucleic acid (RNA) sequencing of the tumor rather than a pathological examination and eliminates the subjective nature of morphology review. It also leads to more effective treatment regimens specifically chosen based on the tumor progenitor rather than the pathology. Additionally, biomarker profiling allows earlier detection and diagnosis and reduction in both false positive sampling rates and misdiagnosis.

Samples for biomarker profiling are collected through either diagnostic surgery or diagnostic needle biopsy. The former is undesirable due to the need for general anesthesia, inpatient care, and increased costs and complications. Diagnostic needle biopsies are performed through fine needle aspiration (FNA) or CNB. CNB provides a larger tissue sample block than FNA and is desirable when trying to extract the increased material volume necessary for biomarker profiling. The CNB procedure is generally performed by radiologists under the guidance of technologies such as but not limited to ultrasound imaging or computed tomography to determine when the needle has reached the targeted tumor mass.

Though these guidance methods have significantly improved successful biopsy rates, the effectiveness of tumor detection is still unacceptably low; the failure rate for acquiring adequate prostate tumor samples is 25-75%. This failure rate is due to limitations of the modalities to provide proper resolution and contrast during the CNB procedure. Examples include cases where a tumor does not have defined architecture and edges, a benign lesion mimics malignancy, or the anatomical site is difficult to access (e.g., the axillary region or prostate gland). Because there is no in situ confirmation of tumor prior to capture, issues such as registration misalignment between needle and image are only realized by inferior tissue samples identified during pathology. Therefore, to collect enough material, health care providers can take from 3-12 cores in more conventional procedures to 60-80 cores in a more comprehensive transperineal saturation biopsy technique.

Regional Anesthesia (RA) requires inserting a sharp cannula through delicate anatomy until the distal tip approaches the targeted neural structure. RA is divided into two main categories—Central, where the spinal nerves/cord are targeted and Peripheral, where a specific nerve bundle is targeted.

Central:

Epidural anesthesia requires inserting a needle (e.g., 17G (gauge)) through the tough ligament and muscle of the back and into the epidural space. After the distal tip reaches the epidural space, the catheter is threaded through the needle. The greatest risks from epidural needle insertion are puncturing the dural membrane and nerve injury, due to the tough ligamentum flavum that is just proximal to the epidural space (i.e., a potential space) and softer dura. Incorrect trajectory and bone contact can create more pain for the patient and increased time for the procedure. The challenge is to provide a method of tissue discrimination anterior to the needle for earlier identification of a) mis-trajectory and b) tissue type/thickness with minimal signal contamination from bony structures (vertebrae).

Peripheral:

Peripheral anesthesia requires inserting a needle (e.g., 18G) through the tissue layers, until the distal tip of the needle is close to the target nerve(s) or nervovascular bundle, without damaging the nerve by intra-neural injection. Peripheral anesthesia has shown an advantage over Central anesthesia due to decreased hospital length of stay and superior pain control with fewer side-effects. Ultrasound imaging is often used to guide the needle tip close to the nerve; however, precise in-plane needle tip localization within various tissue layers remains a challenge. Chronic pain management uses fluoroscopy to guide needle placement, exposing the patient and health care provider to harmful radiation.

Photoacoustic (PA) imaging is a fundamental shift in how tissue composition can be characterized. A short laser pulse is directed into biological tissue where the thermal absorption is highly dependent on the chemical composition of the tissue structures. Because the pulse is shorter than the thermal and elastic relaxation times of biological tissues, this absorption ultimately results in acoustic (ultrasound) generation that can be detected by a separate sensor. Sensing of tissue type and enhanced tissue contrast is superior compared to conventional ultrasound imaging because the modality is not based purely on mechanical properties of the tissue (i.e., density and sound velocity). High spatial resolution and sensitivity are possible because of the one-way (transmitter-to-tissue) light propagation, which provides less attenuation and scattering of light relative to purely optical (two-way propagation) techniques.

Typical PA systems use large, powerful, and costly Q-switched laser sources to create very high intensity beams which are then diverged to illuminate an area of tissue, often several square millimeters at the surface. This approach is used to ensure that an adequate fluence to produce detectable PA signals is achieved over the whole area. Fluence ranges have varied between investigators from approximately 1.4 mJ/cm² to 20 mJ/cm² (the clinical exposure threshold limit at short wavelengths). Resolution of the systems is based on the laser pulse width and resonance frequency of the ultrasound receiver. The Q-switched laser sources generally provide pulse widths of 5 ns to 10 ns. The ultrasound receivers in these systems are typically linear (phased) ultrasound array imaging systems that incorporate complex beamforming techniques to produce high-resolution, 2D images of the tissues from the PA signals.

By using an interrogation method that is essentially producing a 1D image, or line of data, more focused and lower intensity laser sources can be used. Laser diodes are less costly and require less electronics infrastructure than Q-switched laser sources. This not only allows sources producing multiple laser wavelengths to be housed in a single, practically sized system, but also reduces costs by an order of magnitude. By using an optical fiber with a diameter less than 200 μm (numerical aperture, 0.14-0.22), the illumination area is greatly decreased. Laser diodes of less than 500 mW can achieve a laser fluence of 3 mJ/cm², which is sufficient to produce a PA response. This is in part due to the longer pulse time of diodes relative to other laser sources. This is at the expense of resolution, though research in the UK demonstrated that using laser diodes with pulse widths up to 500 ns could produce adequate PA images.

Multispectral Optoacoustic Imaging may be used for successful tumor interrogation with the present invention. Conventional multispectral imaging is a technique where many images are obtained at discrete wavelengths and then recombined into composite images to highlight and identify features through the resulting color patterns. This can highlight areas such as water, vegetation, or roads in satellite imagery or even different antibodies in mixed immunohistochemical staining. Multispectral photoacoustic imaging (MSPI) is similar in theory; each recorded “image” consists of the time-domain photoacoustic echo that results from discrete wavelength light (laser light) illuminating the structures. Researchers that have so far used MSPI for biological imaging have mainly relied on tunable lasers (laser/oscillator combinations) to provide spectral bandwidth of up to 2200 nm. Using this bandwidth, the lasers have matched multiple absorption peaks of lipids, collagen, and hemoglobin to distinguish plaques, tumors, muscle, and bony structures. Though these are highly flexible systems, the laser sources are large, costly and the wavelength scan speeds are tens of nanometers per second much too slow for real-time imaging with MSPI over an 800 nm bandwidth.

### BRIEF SUMMARY OF THE INVENTION

Various features of the invention will be set forth in part in the following description, or may be obvious from the description, or may be learned from practice of the invention. It is hereby noted that the term “in vivo” is defined as performing an act or process within a living organism or natural setting. For example, performing the act of prostate tissue photoacoustic characterization in vivo refers to illuminating prostate tissue in a living being, human or other, while it is in place and still performing all natural physiological functions.

The device herein may be used in a range of tissue types in vivo in a human or animal. The device may be in some aspects of some exemplary embodiments a control box coupled to a reusable handpiece and a disposable needle system that work together to identify biological tissues and fluids distal to the disposable needle during procedures that include needle insertions into the body. Some embodiments may differentiate healthy and cancer cells in situ. These embodiments may allow repositioning of a needle during biopsies prior to tissue capture to maximize the amount of tissue sample collected. Yet other embodiments may differentiate between tissue types, muscle, spaces (e.g., epidural space), and vessels. This will provide the health care provider with feedback in real-time to allow needle repositioning, improve needle localization and decrease the likelihood of over-insertion. The system may be used in conjunction with a conventional ultrasound imaging system for needle visualization, a method that is currently considered standard protocol for many procedures that involve needle insertions.

The disposable needle system may in some exemplary embodiments consist of both a cannula and stylet with integration of an optical fiber into the stylet to deliver light pulses through the stylet and out of the distal end—allowing materials directly in front of the needle to be illuminated. The cannula and stylet may be separable from one another in some procedures to allow injection or aspiration through the cannula after placement in the body. Integration of the disposable needle system with the reusable handpiece may require the use of custom connections.

The control box may house one or more light sources each of which are capable of very short time duration pulses of light. These pulses of light may provide a short burst of energy that is large enough to produce a photoacoustic effect and short enough to not produce any damage in tissues, biological fluids, or other structures. The use of multiple wavelengths of light provides the ability to distinguish the biological materials based on a multispectral approach, whereby each material exhibits a unique pattern of acoustic signals based on the interaction and absorption of light with the chemical structure of the illuminated materials.

A method is also disclosed in other aspects of other exemplary embodiments for the in vivo photoacoustic distinction of biological tissues or fluids during a needle insertion procedure within a living being. The method may include coupling a first end of a disposable needle system incorporating a fiber optic member to a handpiece where the handpiece remains outside of the living being. A second end of the disposable needle system may be placed through the skin of the living being into sub-dermal tissues. The method may also involve coupling the handpiece to an illumination mechanism where the illumination mechanism produces light output at multiple distinct wavelengths, and energizing the illumination mechanism such that the disposable needle system receives light at the first end and transfers the light out of the second end of the disposable needle system in a distal direction. The light exiting the second end of the disposable needle system may pass into the biological tissues or fluids of the living being to interact in such a way as to produce an acoustic response from the biological tissues or fluids. Further, an acoustic receiver may be positioned on the surface of the living being and interact with proximal-traveling acoustic pressure waves and convert the acoustic pressure waves into voltage or charge signals. The method may also include coupling the acoustic receiver to a receiver mechanism where the receiver mechanism samples the voltage or charge signals and also remains outside of the living being, and binning the sampled amplitudes of the voltage or charge signals from the receiver mechanism for each distinct wavelength at each time point or set of time points. Additionally, the method may involve using an algorithm to compare the combination of all sampled amplitudes at each time point or set of time points with combinations of amplitudes of known biological materials or other materials, producing a prediction of what biological material or other material the unknown materials are at each time point or set of time points, and converting each predicted biological material or other material to a distinct representative color. The method may involve relaying the resultant color line representing the biological materials or other materials as a function of time or distance to a display monitor, and the display monitor may remain outside of the living being.

These and other features and aspects of the present invention will become better understood with reference to the following description and appended claims. The accompanying drawings, which are incorporated in and constitute part of this specification, illustrate embodiments of the invention and, together with the description, serve to explain the principles of the invention.

### BRIEF DESCRIPTION OF SEVERAL VIEWS OF THE DRAWINGS

A full and enabling disclosure of the present invention, including the best mode thereof, directed to one of ordinary skill in the art, is set forth more particularly in the remainder of the specification, which makes reference to the appended Figs. in which:

FIG. 1 shows a basic diagram of the components of an embodiment of a photoacoustic needle insertion platform.

FIG. 2A illustrates a more detailed diagram of optomechanical and electrical components within an embodiment of a control box for an integrated photoacoustic needle insertion platform.

FIG. 2B illustrates a more detailed diagram of optomechanical and electrical components within an alternate embodiment of a control box for an integrated photoacoustic needle insertion platform with separate laser coupling circuit.

FIG. 3 shows an embodiment of an ultrasound receiver adhesive patch for receiving photoacoustic signals emanating from within tissue at the skin surface.

FIG. 3A is a cross-sectional view taken along line A-A of FIG. 3.

FIG. 4 is a top plan view of an embodiment of the distal end of an optical biopsy needle with integrated optical fiber for use with an integrated photoacoustic needle insertion platform.

FIG. 4A is a cross-sectional view taken along line A-A of FIG. 4.

FIG. 4B is a perspective view of an embodiment of the distal end of an optical biopsy needle with optical fiber protruding from a distal tip of the optical stylet.

FIG. 4C is a perspective view that shows an embodiment of a biopsy gun with integrated optical biopsy needle showing basic internal needle hub connections and connecting optical fiber.

FIG. 5 is a top view of an embodiment of the distal end of a Tuohy optical anesthesia needle with integrated optical fiber for use with an integrated photoacoustic needle insertion platform.

FIG. 5A is a cross-sectional view taken along line A-A of FIG. 5.

FIG. 5B is a detailed perspective view of an embodiment of the distal end of a Tuohy optical anesthesia needle with integrated optical fiber protruding from a small window in the cannula.

FIG. 5C is a detailed perspective view of an embodiment of an anesthesia handpiece with integrated optical anesthesia needle showing Luer connection and connecting optical fiber.

FIG. 6 is a series of graphs that illustrate the principle of biomaterials differentiation using multispectral photoacoustic profiles assembled from the four different laser transmission wavelengths; fat and oxygenated hemoglobin (O2Hb) demonstrate different photoacoustic amplitude spectra.

FIG. 7 is a graph that illustrates the complex wavelength-dependent spectral absorption coefficients of biological tissues and fluids, highlighting significant differences between biomaterials.

FIG. 8A is a side view of the photoacoustic needle insertion platform and associated readout that demonstrates initial insertion of the optical biopsy needle at a non-optimal trajectory for maximal tumor capture during a biopsy procedure.

FIG. 8B is a side view of the photoacoustic needle insertion platform and associated readout that illustrates deeper penetration of the optical biopsy needle and higher confidence of tumor shown on the display monitor as the optical needle nears tumor.

FIG. 8C is a side view of the photoacoustic needle insertion platform and associated readout that illustrates redirection of the optical biopsy needle as the health care provider changes needle angle searching for a region of larger tumor for capture. The increased photoacoustic signal from the larger tumor area is shown on the display monitor.

FIG. 8D is a side view of the photoacoustic needle insertion platform and associated readout that illustrates the optical biopsy needle after trigger and throw for maximal tumor capture.

Repeat use of reference characters in the present specification and drawings is intended to represent the same or analogous features or elements of the invention.

REFERENCE LABELS

| | |
|---|---|
| 1 | Control Box |
| 2a | Short Wavelength Output |
| | Laser Beam |
| 2b | Medium Wavelength Output |
| | Laser Beam |
| 2c | Long Wavelength Output |
| | Laser Beam |
| 2d | Extra-Long Wavelength Output |
| | Laser Beam |
| 3 | Dichroic Mirrors |
| 4a | Short Wavelength Laser Diode |
| 4b | Medium Wavelength Laser Diode |
| 4c | Long Wavelength Laser Diode |
| 4d | Extra-Long Wavelength Laser Diode |
| 5 | Photoacoustic Needle Insertion Platform |
| 6 | Fiber Optic Coupler |
| 7 | Transfer Optical Fiber |
| 8 | Handpiece |
| 9a | Short Wavelength Driver |
| 9b | Medium Wavelength Driver |
| 9c | Long Wavelength Driver |
| 9d | Extra-Long Wavelength Driver |
| 10 | Coupling Circuit |
| 11 | Optical Shutter |
| 12 | Acoustic Receiver Patch |
| 13 | Preamplifier Circuit |
| 14 | Multi-Pin Connector |
| 15 | Multi-Line Cable |
| 16 | Inner Diameter |
| 17 | Outer Diameter |
| 19 | Piezoelectric Polymer Film |
| 20 | Electronics Sub-systern |
| 21 | Optomechanics Sub-system |
| 22 | Adhesive Film |
| 23 | Power Supply |
| 24 | Power Cable |
| 25 | Display Monitor |
| 26 | Nonconductive Protective Film |
| 27 | Biopsy Gun |
| 28 | Optical Biopsy Needle |
| 29 | Optical Stylet |
| 30 | Stylet Hub |
| 31 | Biopsy Cannula |
| 32 | Cannula Hub |
| 33 | Stylet Trigger Post |
| 34 | Cannula Trigger Post |
| 35 | Embedding Matrix |
| 36 | Stylet Sample Notch |
| 38 | Needle Optical Coupler |
| 39 | Stylet Optical Fiber |
| 40 | Handpiece Optical Coupler |
| 41 | Anesthesia Handpiece |
| 42 | Optical Anesthesia Needle |
| 43 | Anesthesia Stylet |
| 44 | Anesthesia Cannula |
| 45 | Stylet Coupler |
| 46 | Anesthesia Cannula Hub |
| 47 | On/Off Power Button |
| 48 | Skin Surface |
| 49 | Coaxial Output Laser Beam |
| 50 | Tumor |
| 51 | Wireless Transmitter |

### DETAILED DESCRIPTION OF REPRESENTATIVE EMBODIMENTS

Reference will now be made in detail to embodiments of the invention, one or more examples of which are illustrated in the drawings. Each example is provided by way of explanation of the invention, and not meant as a limitation of the invention. For example, features illustrated or described as part of one embodiment can be used with another embodiment to yield still a third embodiment. It is intended that the present invention include these and other modifications and variations.

It is to be understood that the ranges mentioned herein include all ranges located within the prescribed range. As such, all ranges mentioned herein include all sub-ranges included in the mentioned ranges. For instance, a range from 100-200 also includes ranges from 110-150, 170-190, and 153-162.

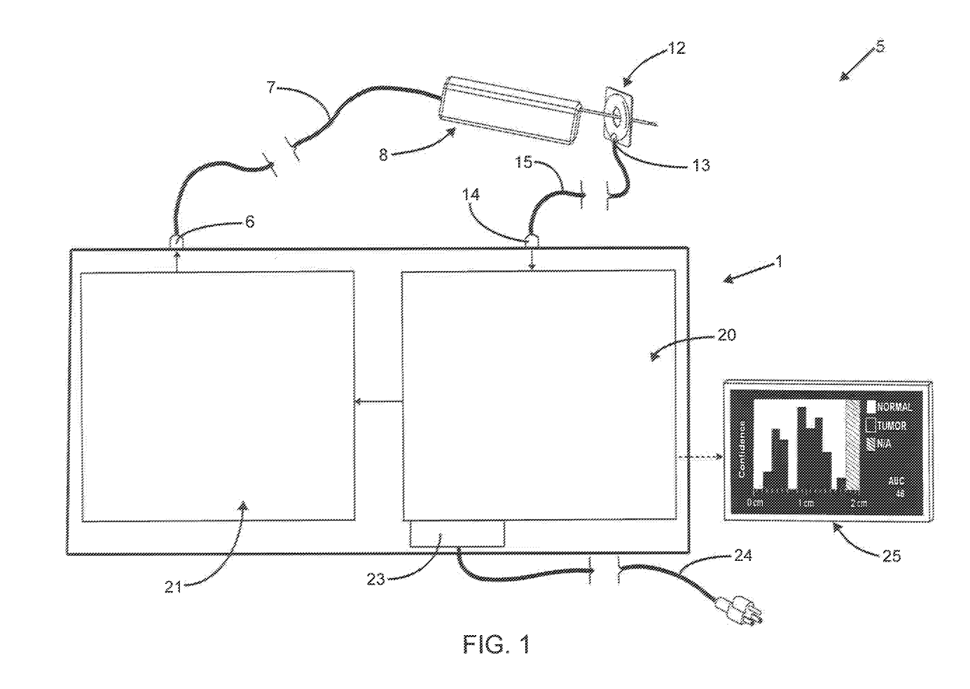

The present photoacoustic needle insertion devices and methods may provide a means to differentiate biological tissues and fluids, such as but not limited to muscle, fat, bone, nerves, deoxygenated or oxygenated blood, and tumorous or necrosed tissue, directly along the projected trajectory of a needle or similar lancing device during medical diagnostic or treatment procedures or examinations using needles, preferably a regional anesthesia, biopsy or vascular access procedure. Implementing the light pulses into a needle system may require, in some instances, the use of custom connections. Certain preferred embodiments are illustrated in FIGS. 1-8D with the numerals referring to like and corresponding parts.

As used herein, the distal direction is the direction toward the patient and away from the health care provider. The proximal direction is toward the health care provider and away from the patient. Illustrations used herein are specific to four laser sources but the number of laser diode sources, and therefore the number of interrogation wavelengths, could be reduced or increased with modification in accordance with various exemplary embodiments.

FIG. 1 illustrates a basic diagram of an embodiment of an integrated photoacoustic needle insertion platform 5, which is comprised of four main sub-systems: a control box 1 that houses the electronics sub-system 20 and optomechanics sub-system 21; the needle insertion handpiece 8; the acoustic receiver patch 12; and display monitor 25. The photoacoustic needle insertion platform 5 works by producing light in the optomechanics sub-system 21, transmitting the light through the needle insertion handpiece 8 and into the subject, and receiving acoustic echoes through the separate acoustic receiver patch 12 and displaying the multispectral photoacoustic tissue information on the display monitor 25. The electronics are powered by a power supply 23 that is connected to a conventional wall outlet such as but not limited to between 100-240 V, 50-60 Hz with a power cable 24. The display monitor 25 provides a representation of the tissue types or confidence of tissue types directly ahead of the needle insertion handpiece 8. The needle insertion handpiece 8 connects to the control box 1 through a transfer optical fiber 7 with a fiber optic coupler 6 on the proximal end. The acoustic receiver patch 12 is connected to the control box 1 through a multi-line cable 15 with a multi-pin connector 14 on the proximal end. A preamplifier circuit 13 on the acoustic receiver patch 12 is powered through the multi-line cable 15 with a direct current voltage such as but not limited to between 3 V and 12 V.

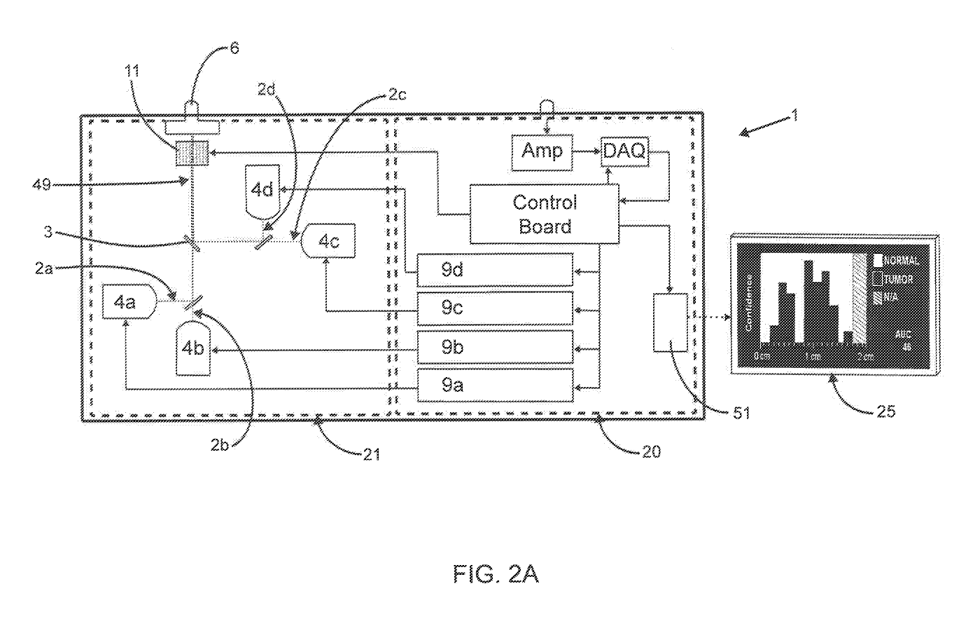

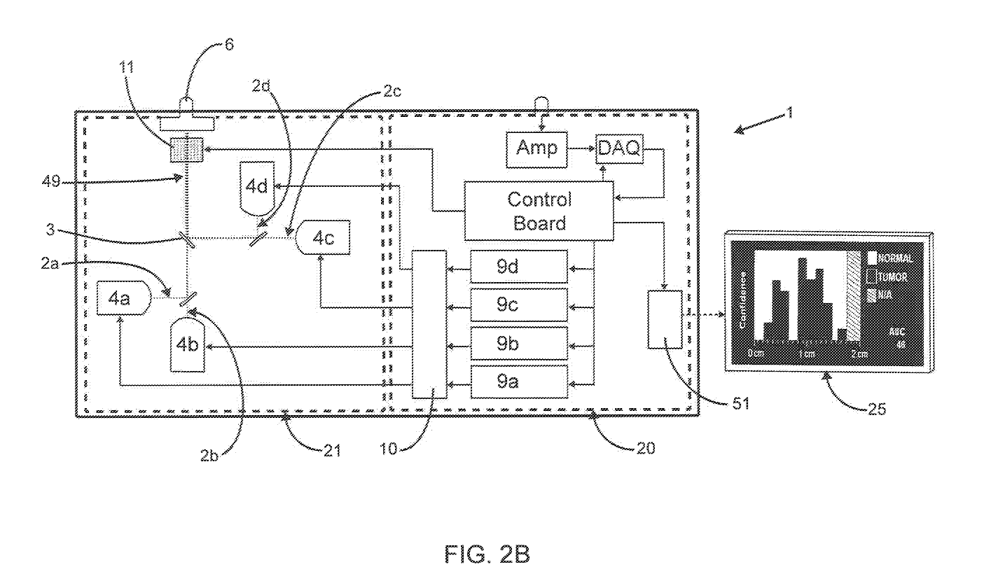

FIG. 2A illustrates a more detailed diagram of an embodiment of the control box 1 and components within the electronics sub-system 20 and optomechanics sub-system 21. A short wavelength laser diode 4a, medium wavelength laser diode 4b, long wavelength laser diode 4c, and extra-long wavelength laser diode 4d, are located within the optomechanics sub-system 21. The use of multiple wavelengths of light provides the ability to distinguish biological materials based on a multispectral approach, whereby the short wavelength laser diode 4a produces a short wavelength output laser beam 2a, which is shorter (smaller) than the medium wavelength output laser beam 2b output from the medium wavelength laser diode 4b. The long wavelength output laser beam 2c from the long wavelength laser diode 4c is longer (larger) than the medium wavelength output laser beam 2b. The extra-long wavelength output laser beam 2d from the extra-long wavelength laser diode 4d is the longest (largest) wavelength of the optomechanics sub-system 21. The wavelengths of the output laser beams 2a-2d of the laser diodes 4a-4d are all within the light spectrum from 250 nm to 1800 nm, but preferentially between 450 nm and 1300 nm. The optical power of the output laser beams 2a-2d of the laser diodes 4a-4d are all within the range of 25 mW to 10 W, but preferentially between 100 mW and 2 W.

The output laser beams 2a-2d may or may not be collimated, or have minimal diffraction, due to focusing. The output laser beams 2a-2d from the laser diodes 4a-4d are directed through a series of dichroic mirrors 3 that are reflective or transmissive to specific light wavelengths such that all of the output laser beams 2a-2d form a single coaxial output laser beam 49 that enters a fiber optic coupler 6, such as a focused aspheric lens, after passing through a controllable optical shutter 11 that is used to block any output for safety during non-use. The laser diodes 4a-4d are controlled by a short wavelength driver 9a, medium wavelength driver 9b, long wavelength driver 9c, and extra-long wavelength driver 9d that create pulses of electrical current at least equal in magnitude to the emission threshold current but less than 110% of the maximum operating current of the laser diodes 4a-4d. The time durations of the electrical current pulses, as defined by the full width at half maximum time duration, are between 1 nanoseconds (ns) and 500 ns, but preferentially between 30 ns and 150 ns. During each pulse cycle, each laser diode 4a-4d is driven by a single electrical current pulse by the respective laser diode driver 9a-9d. The time delay between single electrical current pulses to the laser diodes 4a-4d, such as the time delay between pulsing laser diode 4a with laser diode driver 9a and pulsing laser diode 4b with laser diode driver 9b, are between 1 microsecond and 400 microseconds, but preferentially between 10 microseconds and 100 microseconds. The time duration of the pulse cycle is between 5 microseconds and 10 milliseconds (ms), but preferentially between 250 microseconds and 2 ms. The display monitor 25 communicates with the electronics sub-system 20 through a wireless protocol and wireless transmitter 51, and is powered by batteries. In a less preferential embodiment, the display monitor 25 is physically connected to the control box 1 and communicates with the electronics sub-system 20 through a hardwired connection.

FIG. 2B illustrates an alternate embodiment of the control box 1 and components within the electronics sub-system 20 and optomechanics sub-system 21 (shown in FIG. 2A), in which the laser diode drivers 9a-9d provide pulses of electrical current or direct current electrical current that are 10-99% of the magnitude of the emission threshold current of the laser diodes 4a-4d, but preferentially 75-95% of the emission threshold current of the laser diodes. A separate coupling circuit 10 within the control box 1 provides current pulses to the laser diodes 4a-4d via an incorporated coupling capacitor and biasing electronics, such that the sum total current to each laser diode 4a-4d exceeds the emission threshold current and is less than 110% of the maximum operating current for the respective laser diode 4a-4d. This configuration provides the ability to emit pulses with a finer control than the first configuration (in FIG. 2A) by using the separate coupling circuit 10 to provide smaller and quicker pulses in electrical current to the laser diodes 4a-4d. The laser diode drivers 9a-9d in this case provide a ‘priming’ effect to place the laser diodes 4a-4d at excitation states just below laser emission. The time durations of the electrical current pulses from the coupling circuit 10, as defined by the full width at half maximum time duration, are between 1 ns and 500 ns, but preferentially between 30 ns and 150 ns. The time duration of the electrical current from the laser diode drivers 9a-d may be as short as 1 ns and may be as long as the integrated photoacoustic needle insertion platform 5 is powered if a direct current is used. The coupling circuit 10 provides single electrical current pulses to the laser diodes 4a-4d with time delays between 1 microsecond and 400 microseconds, but preferentially between 10 microseconds and 100 microseconds. The time duration of the pulse cycle defined by the coupling circuit 10 is between 5 microseconds and 10 ms, but preferentially between 250 microseconds and 2 ms. The display monitor 25 communicates with the electronics sub-system 20 through a wireless protocol and wireless transmitter 51, and is powered by batteries. In a less preferential embodiment, the display monitor 25 is physically connected to the control box 1 and communicates with the electronics sub-system 20 through a hardwired connection.

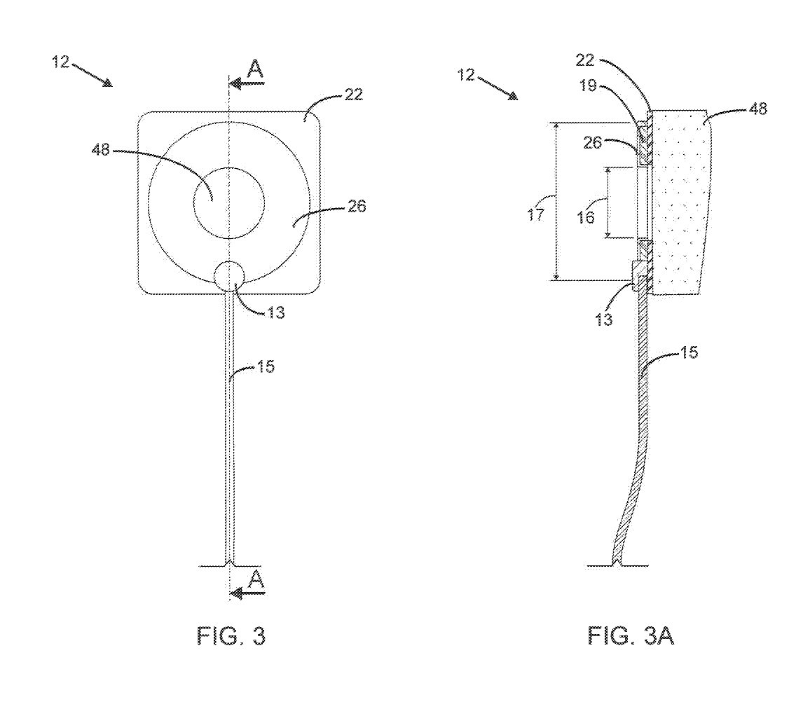

FIGS. 3 and 3A illustrates in more detail the components of the acoustic receiver patch 12 that receives acoustic pressure waves from within the patient or subject. The active portion of the acoustic receiver patch 12 consists of a piezoelectric polymer film 19 made from a material such as poly(vinylidene-difluoride) or its copolymer or, less preferentially, an active piezoelectric ceramic or single crystal material. The thickness of the piezoelectric polymer film 19 is between 9 microns and 200 microns, but preferentially between 20 microns and 52 microns. The piezoelectric polymer film 19 may be of an annular geometry and may be comprised of a single electrical element or, less preferably, may be comprised of multiple electrical members. In an alternate less preferential embodiment, the piezoelectric polymer film 19 may be of a disk, rectangular or other geometry and may be comprised of a single or multiple electrical members. The inner diameter 16 of the piezoelectric polymer film 19 is in the range of 3 mm to 25 mm, but preferentially in the range of 8 mm to 15 mm. The outer diameter 17 of the piezoelectric polymer film 19 is a dimensional range of 1 mm to 10 mm larger than the inner diameter 16, but preferentially in the range of 10 mm to 20 mm. The other layers of the acoustic receiver patch 12 consist of electrically conductive tin/silver layers covering both faces of the piezoelectric polymer film 19, an adhesive film 22, preferably made from biocompatible materials, for bonding to the skin surface 48 of the patient or subject, and a non-conductive protective film 26, preferably made from polyimide. All layers may be bonded with a non-conductive epoxy or similar material, preferably a flexible epoxy. A preamplifier circuit 13 bonded to the acoustic receiver patch 12 provides an initial voltage amplification of the received photoacoustic signal and improves signal-to-noise. A multi-line cable 15 provides power from the control box 1 (from FIG. 1) to the preamplifier circuit 13 and routes the photoacoustic signal from the preamplifier circuit 13 to the control box 1. The multi-line cable 15 connects to the control box 1 through a multi pin connector 14 (from FIG. 1).

As will be discussed in detail later, there are two types of needle insertion systems disclosed herein for example but this invention is not limited to only these two types of needle insertion systems. Both examples use the in-needle photoacoustic interrogation principle to exemplify the inventions. Both approaches may use a control box and receiver system. Both approaches may apply light pulses and record acoustic signals transmitted from within the patient or subject.

### Needle Insertion Design 1 (Core Needle Biopsy System)

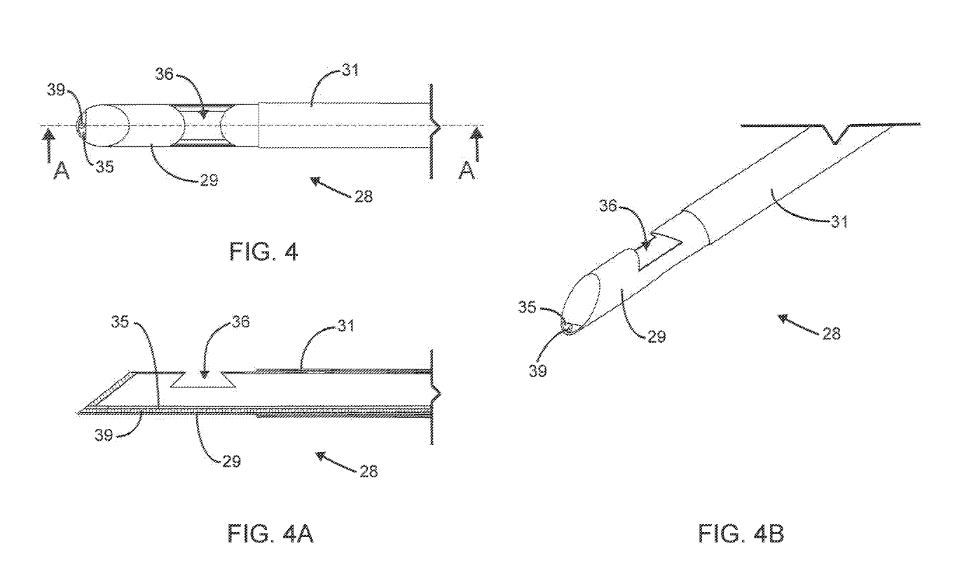

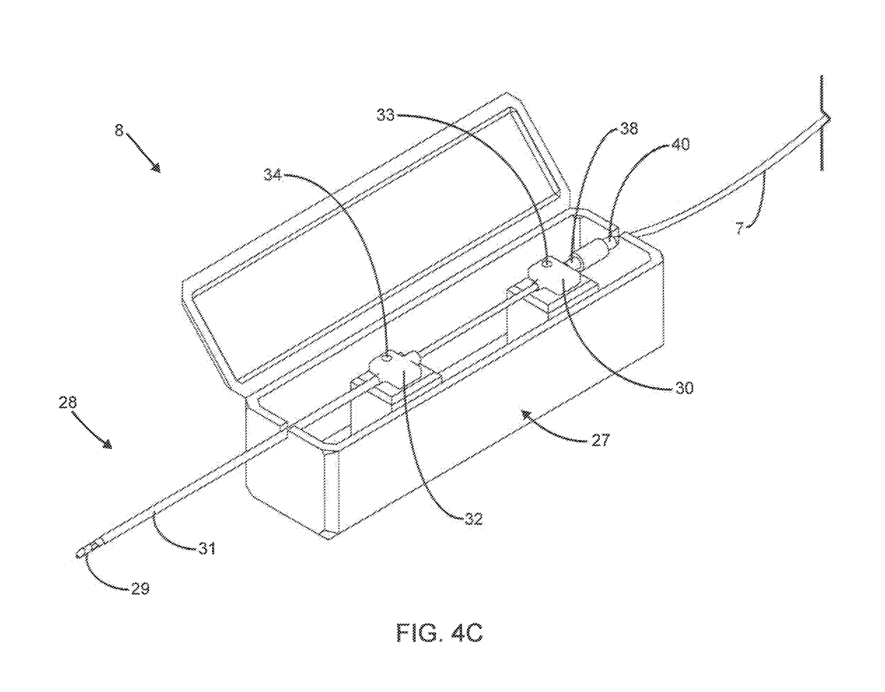

FIGS. 4, 4A, 4B and 4C illustrate in more detail the components of the needle insertion design 1 where the needle insertion handpiece 8 is designed for core needle biopsy and is comprised of a reusable biopsy gun 27, and a disposable optical biopsy needle 28. The disposable optical biopsy needle 28 is comprised of an optical stylet 29 with stylet hub 30, and a biopsy cannula 31 with cannula hub 32. The diameter of the biopsy cannula 31 is in the range of 22 Gauge to 10 Gauge, but preferentially in the range of 18 Gauge to 14 Gauge. FIG. 4A illustrates that the outer diameter of the optical stylet 29 is nearly identical to the inner diameter of the biopsy cannula 31 but allows free linear movement of the two components. The stylet hub 30 on the proximal end of the optical stylet 29 may connect to the biopsy gun 27 via a stylet trigger post 33 to mechanically couple the optical stylet 29 to the trigger and throw mechanism of the biopsy gun 27. The cannula hub 32 on the proximal end of the biopsy cannula 31 may connect to the biopsy gun 27 via a cannula trigger post 34 to mechanically couple the biopsy cannula 31 to the trigger and throw mechanism of the biopsy gun 27. A stylet optical fiber 39 runs through the length of the optical stylet 29 and is secured in an embedding matrix 35. The embedding matrix 35 could be a biocompatible epoxy, but could be other materials. The stylet sample notch 36 is of a typical conventional core needle biopsy sample notch, but the stylet optical fiber 39 is maintained within the embedding matrix 35 below the stylet sample notch 36. The stylet hub 30 contains a through-hole that allows the stylet optical fiber 39 to pass through it, and includes a needle optical coupler 38. The needle optical coupler 38 engages with a handpiece optical coupler 40 that is integrated into the biopsy gun 27, which facilitates good light transmission between the transfer optical fiber 7 and stylet optical fiber 39 while allowing the biopsy gun 27 to be separated from other components for re-sterilization before re-use. When the biopsy gun 27 is triggered, the biopsy cannula 31 and cannula hub 32, optical stylet 29, stylet hub 30, and needle optical coupler 38 are thrown in the distal direction, disengaging the needle optical coupler 38 from the handpiece optical coupler 40.

### Needle Insertion Design 2 (Regional Anesthesia System)

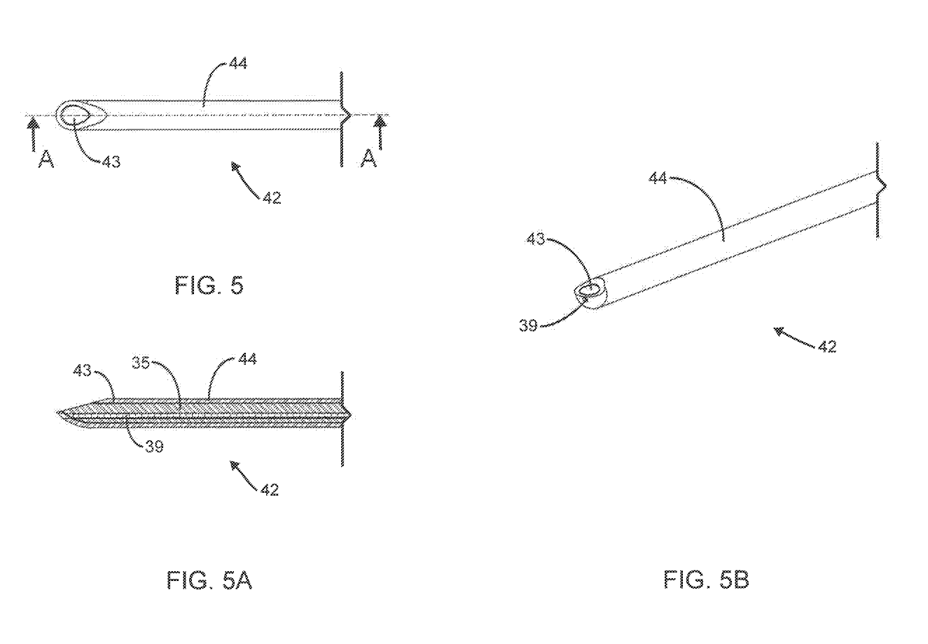

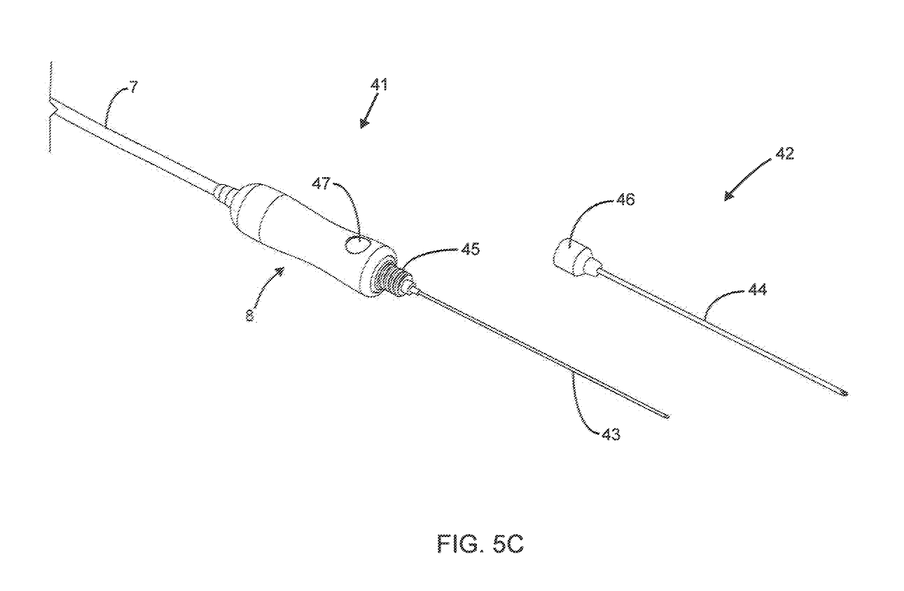

FIGS. 5, 5A, 5B and 5C illustrate in more detail the components of the needle insertion design 2 where the needle insertion handpiece 8 is designed for regional anesthesia delivery and is comprised of a reusable anesthesia handpiece 41, and a disposable optical anesthesia needle 42. The disposable optical anesthesia needle 42 may be but not limited to a Tuohy needle with an anesthesia stylet 43 and anesthesia cannula 44, though other relevant needle designs could be used. The diameter of the anesthesia cannula 44 is in the range of 30 Gauge to 14 Gauge, but preferentially in the range of 22 Gauge to 18 Gauge. FIG. 5 illustrates a small hole or window at the distal tip of the anesthesia cannula 44 that allows the laser light to exit the optical anesthesia needle 42 and illuminate the tissues within the body. The outer diameter of the anesthesia stylet 43 is nearly identical to the inner diameter of the anesthesia cannula 44 but allows free linear movement of the two components. A stylet optical fiber 39 runs through the length of the anesthesia stylet 43 and is secured in an embedding matrix 35. The embedding matrix 35 may be but not limited to a biocompatible epoxy materials. A stylet coupler 45 on the proximal end of the anesthesia stylet 43 connects to the needle insertion handpiece 8 and couples light from the transfer optical fiber 7 into the stylet optical fiber 39. The outside of the stylet coupler 45 incorporates a Luer-lock connector, though a Luer connector or other type of connector could also be used. The anesthesia cannula hub 46 may be but not limited to a corresponding Luer or Luer-lock form factor to facilitate connection to a syringe for injection once the optical anesthesia needle 42 is located correctly and the anesthesia handpiece 41 and anesthesia stylet 43 are removed. An optional on/off power button 47 enables or disables the laser output; in other embodiments, enabling or disabling the laser output could be performed using a foot pedal or switch on the control box 1 (from FIG. 1).

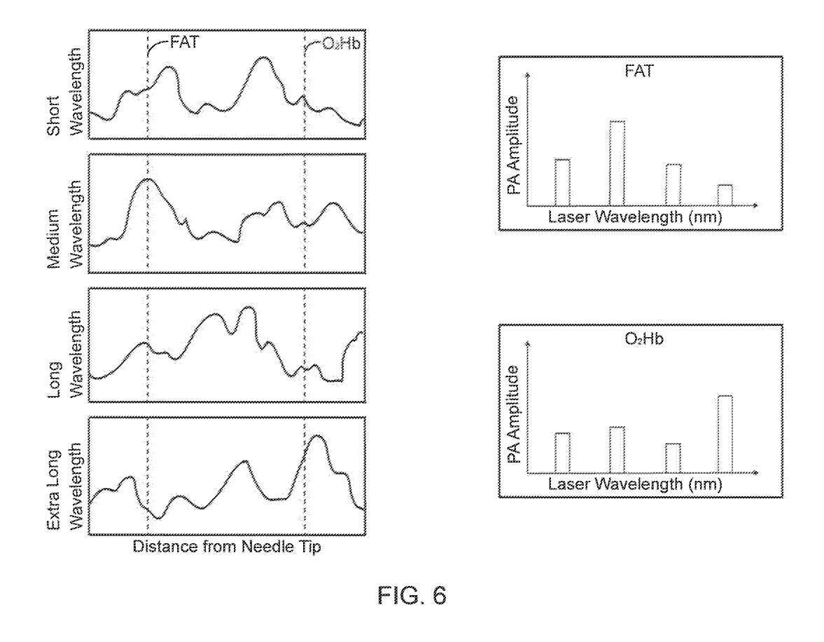

FIG. 6 refers to a method of multispectral photoacoustic interrogation using the photoacoustic needle insertion platform 5 (from FIG. 1). The acoustic measurements recorded after illumination of the tissue with all of the laser wavelengths are evaluated for amplitude. This is preferentially performed after full wave rectification and enveloping of the measurement signals, typical with ultrasound imaging, but other processing may be performed. The set of four acoustic amplitudes for each time point, which is correlated to the distance from the needle tip based on the speed of sound in tissue, are binned together and compared to saved data from known tissue samples within non-volatile memory in the electronics sub-system 20 (from FIG. 1).

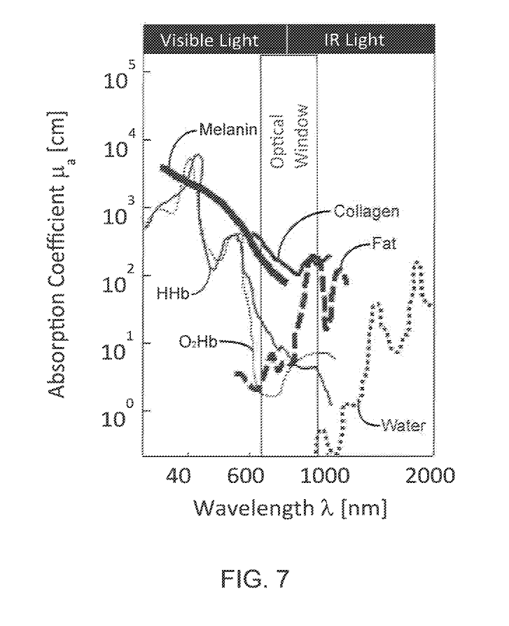

FIG. 7 illustrates the absorption coefficient of different biological materials for wavelengths of light. Because each material exhibits a different absorption spectrum, different biological materials can be differentiated from one another.

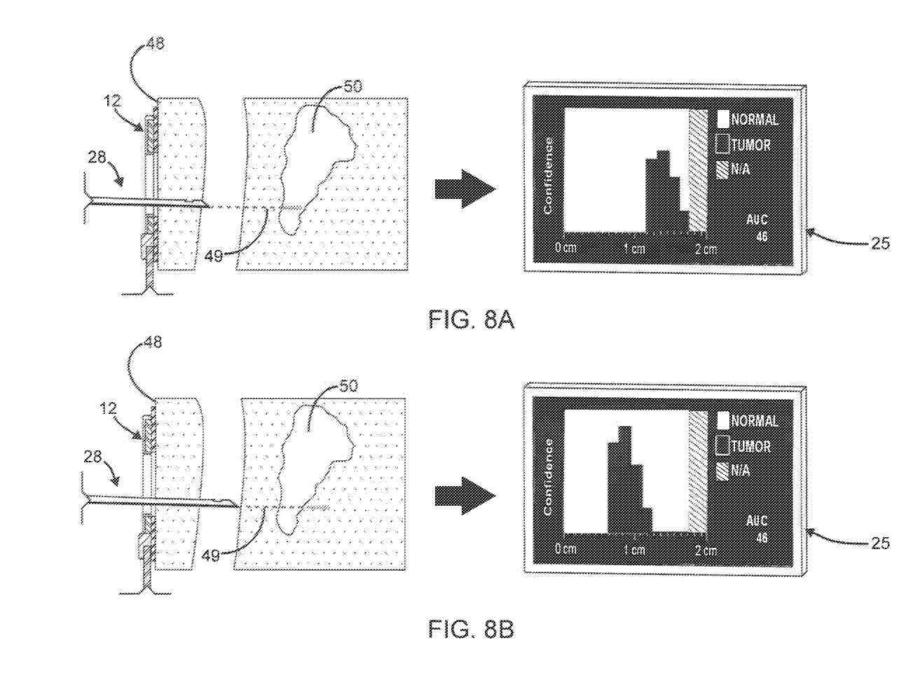

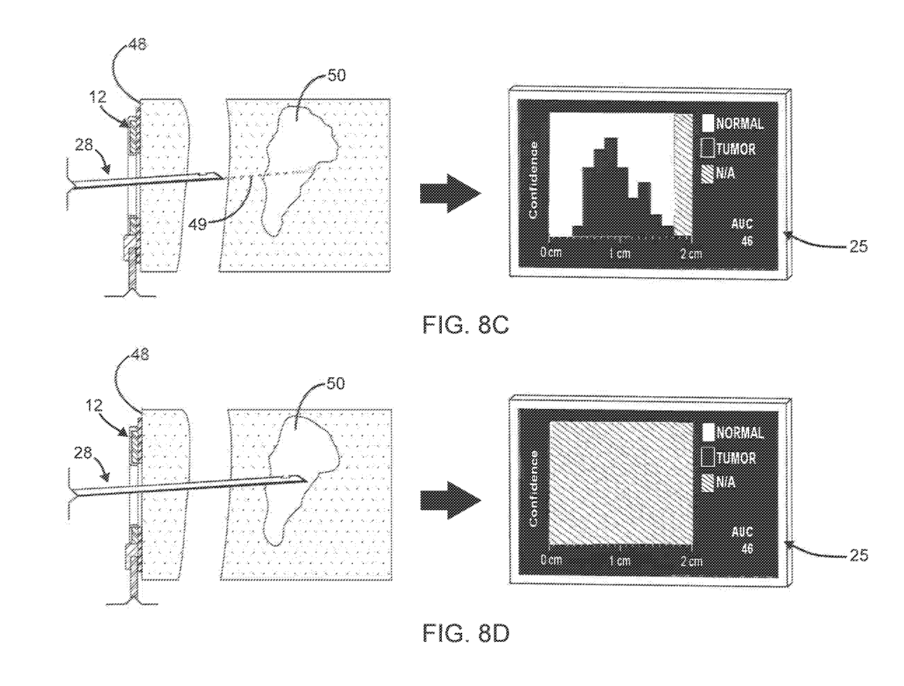

FIGS. 8A-D illustrates an example of the needle insertion process with the invention demonstrating redirection of the optical biopsy needle 28 during a biopsy procedure. As the optical biopsy needle 28 penetrates the skin surface 48 and superficial layers of tissue (in FIG. 8A), photoacoustic signals produced from the coaxial output laser beam 49 are received by the acoustic receiver patch 12. The confidence of a tumor 50 distal to the needle near the 2 cm limit of the coaxial output laser beam 49 is displayed on the display monitor 25. In FIG. 8B, as the optical biopsy needle 28 progresses to deeper tissue layers, the tumor 50 is seen closer to the optical biopsy needle 28 on the display monitor 25. FIG. 8C illustrates realignment of the optical biopsy needle 28 as a health care provider searches for the most viable trajectory for maximal sample capture. When there is confidence of a larger tumor capture along the realigned needle trajectory, FIG. 8D demonstrates throw of the optical biopsy needle 28 along a redirected trajectory relative to the initial trajectory.

While the present invention has been described in connection with certain preferred embodiments, it is to be understood that the subject matter encompassed by way of the present invention is not to be limited to those specific embodiments. On the contrary, it is intended for the subject matter of the invention to include all alternatives, modifications and equivalents as can be included within the spirit and scope of the following claims.

What is claimed:

1. A device for differentiating tissue, comprising:
a first laser transmission source that outputs a first laser beam, wherein output from the first laser transmission source is transferred into the tissue;
a second laser transmission source that outputs a second laser beam that has a wavelength that is different than the first laser beam, wherein output from the second laser transmission source is transferred into the tissue;
a needle system for insertion into the tissue, and;
an acoustic receiver that receives acoustic waves that are created upon the transfer of the output of the first and second laser transmission sources into the tissue.

2. The device as set forth in claim 1, further comprising:
a control box that has the first and second laser transmission sources;
a handpiece that houses a portion of the needle system;
a transfer optical fiber that couples the control box to the handpiece, wherein the output from the first and second laser transmission sources is transferred through the transfer optical fiber to the handpiece and then to the needle system; and
a monitor that displays information about the tissue at a location distal to a terminal distal end of a needle tip of the needle system.

3. The device as set forth in claim 1, wherein the first laser transmission source and the second laser transmission source are laser diodes, wherein the wavelength of the first laser beam is at least 10 nanometers different than the wavelength of the second laser beam, wherein both the first and second laser beams are within the optical spectrum of 450 nanometers to 1300 nanometers.

4. The device as set forth in claim 1, wherein the first and second laser transmission sources produce the first and second laser beams in laser light pulses less than 200 nanoseconds in duration.

5. The device as set forth in claim 4, wherein the first and second laser transmission sources produce the first and second laser beams through direct current pulses.

6. The device as set forth in claim 4, wherein the first and second laser transmission sources produce the first and second laser beams through a current controlled direct current pulse that is applied directly to the transmission source or is applied through a coupling capacitor with biasing electronics.

7. The device as set forth in claim 1, wherein the acoustic receiver is selected from the group consisting of a piezoelectric polymer, a piezoelectric ceramic, a piezoelectric single crystal, and an optoacoustic transducer.

8. The device as set forth in claim 1, wherein the acoustic receiver is arranged as a patch that has an adhesive film, wherein the acoustic receiver has a piezoelectric polymer film that has an annulus shape, wherein the needle system is located through the piezoelectric polymer film.

9. The device as set forth in claim 1, further comprising:
an optomechanics sub-system, wherein the first laser beam and the second laser beam are aligned into one single coaxial beam path; and
a fiber optic coupler that receives and focuses the single coaxial beam path to a proximal end of a single transfer optical fiber.

10. The device as set forth in claim 9, wherein the optomechanics sub-system has a plurality of dichroic mirrors positioned at angles relative to the first and second laser transmission sources such that the first and second laser beams are reflected into the single coaxial beam path that is received by the fiber optic coupler.

11. A device for differentiating tissue, comprising:
a needle system for insertion into the tissue;
an optical fiber carried by the needle system, wherein an output laser beam exits the optical fiber and is directed into the tissue; and
an acoustic receiver that receives acoustic waves that are created upon the transfer of the output laser beam into the tissue.

12. The device as set forth in claim 11, further comprising:
a control box that includes a first laser transmission source and a second laser transmission source that are both diodes, wherein the first laser transmission source outputs a first laser beam, and wherein the second laser transmission source outputs a second laser beam that has a wavelength that is different than the first laser beam, wherein the first and second laser beams are transferred through a fiber optic coupler of the control box;
a transfer optical fiber in communication with the fiber optic coupler that receives the first and second laser beams, wherein output from the fiber optic coupler is transferred through the transfer optical fiber;
a handpiece that houses a portion of the needle system, wherein the transfer optical fiber is coupled to the handpiece, wherein output from the transfer optical fiber is transferred to the handpiece, wherein the handpiece is in communication with the optical fiber, wherein output from the handpiece is transferred to the optical fiber;
wherein output from the acoustic receiver is transferred to the control box;
a monitor in communication with the control box that displays information about the tissue.

13. The device as set forth in claim 11, further comprising:
a handpiece;
wherein the needle system has an optical stylet, wherein the optical fiber is connected to the optical stylet by an embedding matrix;
wherein the needle system has a biopsy cannula through which the optical stylet is disposed, wherein the optical stylet moves relative to the biopsy cannula;
wherein the needle system has a stylet hub that connects a proximal end of the optical stylet to the handpiece; and
wherein the needle system has a cannula hub that connects a proximal end of the biopsy cannula to the handpiece.

14. The device as set forth in claim 13, wherein the handpiece has a trigger mechanism that when triggered moves the optical stylet, the biopsy cannula, the stylet hub, and the cannula hub in a distal direction relative to the handpiece.

15. The device as set forth in claim 14, wherein the handpiece has a handpiece optical coupler, and wherein the needle system has a needle optical coupler, wherein when the trigger mechanism is triggered the needle optical coupler moves in the distal direction relative to the handpiece, wherein the handpiece optical coupler engages the needle optical coupler and wherein the needle optical coupler receives output from the handpiece optical coupler.

16. The device as set forth in claim 13, wherein the stylet hub is aligned with a stylet post of the handpiece, and wherein the cannula hub is aligned with a cannula post of the handpiece.

17. The device as set forth in claim 11, further comprising:
a handpiece;
wherein the needle system has an anesthesia stylet that is coupled to a distal end of the handpiece by a stylet coupler, wherein the optical fiber runs through the anesthesia stylet and is connected to the anesthesia stylet by an embedding matrix disposed within the anesthesia stylet;
wherein the needle system has an anesthesia cannula carried by the handpiece, wherein the anesthesia stylet is disposed through the anesthesia cannula.

18. The device as set forth in claim 11, wherein the optical fiber is oriented along a length axis of a needle of the needle system and wherein the output laser beam exits a distal end of the needle and travels in a path nominally equal to a physical trajectory of the needle; and further comprising a monitor that displays information about the tissue at a location distal to a terminal distal end of the needle.

19. A method for identifying different tissue types, comprising the steps of:
inserting a needle with an optical fiber into biological tissue;
transmitting an output laser beam out of the optical fiber and into the biological tissue, wherein the output laser beam is a series of light pulses that have different wavelengths;
recording photoacoustic echoes from the biological tissue after each light pulse;
using the photoacoustic echoes from at least a subset of the wavelengths to produce photoacoustic signatures over a range of depths;
using the photoacoustic signatures to compare with prior collected data of known biological tissues to differentiate the biological tissue; and
displaying depth-dependent, differentiated tissue data to a user.

20. The method as set forth in claim 19, wherein the photoacoustic signatures are based on a measurement selected from the group consisting of time-domain voltage amplitudes, and frequency-domain spectral amplitudes from the photoacoustic echoes measured by an acoustic receiver.
